(19) United States
(12) Patent Application Publication
(10) Pub. No.: US 2011/0078832 A1
(43) Pub. Date: Mar. 31, 2011

(54) HYDROPHILIC POLYURETHANE COATINGS

(75) Inventors: Jürgen Köcher, Langenfeld (DE); Thorsten Rische, Columbus, GA (US)

(73) Assignee: Bayer MaterialScience AG, Leverkusen (DE)

(21) Appl. No.: 12/995,093

(22) PCT Filed: May 19, 2009
(86) PCT No.: PCT/EP2009/003543

§ 371 Date: Nov. 29, 2010

(30) Foreign Application Priority Data
May 28, 2008 (DE) 102008025613.7

(51) Int. Cl. C09D 175/04; B05D 5/06; B05D 1/36; B32B 17/10; A01H 5/10

(52) U.S. Cl. 800/298; 524/537; 427/165; 427/261; 428/425.6

(57) ABSTRACT

The present invention relates to a coating composition in the form of a dispersion containing a polyurethane urea which (1) is terminated by a copolymer unit of polyethylene oxide and polypropylene oxide, and (2) contains at least one hydroxyl-group-containing polycarbonate polyol.

The present invention relates to the use of a coating composition in the form of a polyurethane dispersion in the production of hydrophilic coatings, in particular to the use of the coating composition in the coating of devices, in particular medical devices. In addition, the hydrophilic coating materials according to the invention can also be used to protect surfaces from condensation, to produce surfaces that are easy to clean or self-cleaning, and to reduce the uptake of dirt by such surfaces. The hydrophilic coating materials according to the invention are additionally capable of reducing or avoiding the formation of water spots on surfaces.

It is further possible with the polyurethane dispersions according to the invention to produce hydrophilic surfaces which no longer become overgrown to a noteworthy extent with organisms that live in water (antifouling properties). Further fields of application of the coating materials according to the invention are applications in the printing industry, for cosmetic formulations as well as for systems, also outside of applications in medical technology, that release active ingredients.

The use of medical devices, for example catheters, can be greatly improved by providing them with hydrophilic surfaces. The insertion and displacement of urinary or blood vessel catheters is simplified because hydrophilic surfaces in contact with blood or urine adsorb a water film. As a result, friction of the catheter surface against the vessel walls is reduced, so that the catheter is easier to insert and move. Direct wetting of the devices before the operation can also be carried out in order to reduce friction through the formation of a homogeneous water film. The patients concerned have less pain, and the risk of damage to the vessel walls is thereby reduced. In addition, when catheters are used in contact with blood there is always the risk that blood clots will form. In this context, hydrophilic coatings are generally regarded as being helpful for antithrombogenic coatings.

Polyurethane coatings prepared from solutions or dispersions of corresponding polyurethanes are suitable in principle for the production of corresponding surfaces.

Thus, U.S. Pat. No. 5,589,563 describes the use of coatings having surface-modified end groups for polymers used in the biomedical field, which polymers can also be used to coat medical devices. The resulting coatings are produced from solutions or dispersions, and the polymeric coatings comprise different end groups which are selected from amines, fluorinated alkanols, polydimethylsiloxanes and amine-terminated polyethylene oxides. However, these polymers do not have satisfactory properties as coatings for medical devices, in particular in respect of the required hydrophilicity.

DE-A 199 14 882 relates to polyurethanes, polyurethane ureas and polyureas in dispersed or dissolved form, which are composed of
(a) at least one polyol component,
(b) at least one di-, tri- and/or poly-isocyanate component,
(c) at least one hydrophilic, non-ionic or potentially ionic chain-extension component consisting of compounds having at least one group reactive towards isocyanate, groups and at least one hydrophilic polyether chain and/or of compounds having at least one group capable of salt formation, which is optionally present in at least partially neutralised form, and at least one group reactive towards isocyanate groups,
(d) at least one chain-extension component other than (a) to (c) having a molecular weight in the range from 32 to 500 and having at least one group reactive towards isocyanate groups, and
(e) at least one monofunctional blocking agent.

The polymer dispersions, which accordingly necessarily contain a monofunctional blocking agent, are used, for example, in sizes.

DE-A 199 14 885 relates to dispersions based on polyurethanes, polyurethane polyureas and polyureas, which are preferably reaction products of
a) at least one polyol component,
b) at least one di-, tri- and/or poly-isocyanate component,
c) optionally at least one (potentially) ionic chain-extension component consisting of compounds having at least one group reactive towards NCO groups and at least one group capable of salt formation, which is optionally present in at least partially neutralised form,
d) optionally at least one non-ionic hydrophilic chain-extension component consisting of compounds that are mono- to tetra-functional within the context of the isocyanate addition reaction and that contain at least one hydrophilic polyether chain,
e) optionally at least one chain-extension component other than a) to d) having a molecular weight in the range from 32 to 2500 and containing groups reactive towards isocyanate groups, and
f) from 0.1 to 15 wt.% of at least one monofunctional blocking agent which consists of at least 50% dimethylpyrazole,

wherein the sum of a) to f) is 100% and wherein either c) or d) cannot be 0 and are used in such an amount that a stable dispersion is formed.

The dispersions are used inter alia in the coating of mineral substrates, in the lacquering and sealing of wood and derived timber products, in the lacquering and coating of metal surfaces, in the lacquering and coating of plastics and in the coating of textiles and leather.

These polyurethane urea dispersions known from the prior art are not used for medical purposes, that is to say for coating medical devices.

In addition, the polyurethane urea coatings known hitherto frequently have disadvantages in that they are not sufficiently hydrophilic to be used as a coating for medical devices.

Within this context, U.S. Pat. No. 5,589,563 recommends surface-modified end groups for biomedical polymers which can be used for coating medical devices. Such polymers comprise different end groups which are selected from amines, fluorinated alkanols, polydimethylsiloxanes and amine-terminated polyethylene oxides. However, such polymers likewise do not have satisfactory properties as coatings for medical devices, in particular in respect of the required hydrophilicity.

Accordingly, it was an object of the present invention to provide polyurethane urea dispersions which can be used to provide or coat medical devices with hydrophilic surfaces. Because these surfaces are frequently used in contact with blood, the surfaces of these materials should also have good blood compatibility and, in particular, reduce the risk of blood clot formation.

This invention therefore provides the use of specific polyurethane urea dispersions in the production of hydrophilic surfaces, as are desirable for providing medical devices and surfaces with antifouling properties.

The polyurethane urea dispersions to be used according to the invention are characterised in that they comprise
(1) at least one polyurethane urea terminated by a copolymer unit of polyethylene oxide and polypropylene oxide, and
(2) at least one polycarbonate polyol.

It has been found that compositions comprising these specific polyurethane ureas are outstandingly suitable as coatings having hydrophilic properties, as are desirable, for example, in many medical devices to improve the insertion properties and at the same time reduce the risk of blood clot formation during treatment with the medical device, and for producing surfaces having antifouling properties, as are desirable, for example, in ship building.

Polyurethane ureas within the scope of the present invention are polymeric compounds comprising
(a) at least two urethane-group-containing structural repeating units having the following general structure

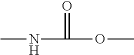

and
(b) at least one urethane-group-containing structural repeating unit

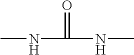

The coating compositions to be used according to the invention are based on polyurethane ureas which have substantially no ionic modification. Within the scope of the present invention this is understood as meaning that the polyurethane ureas to be used according to the invention contain substantially no ionic groups, such as in particular no sulfonate, carboxylate, phosphate or phosphonate groups.

Within the scope of the present invention, the expression “substantially no ionic modification” is understood as meaning that an ionic modification is present in an amount of not more than 2.50 wt. %, preferably not more than 2.00 wt. %, in particular not more than 1.50 wt. %, particularly preferably not more than 1.00 wt. %, especially not more than 0.50 wt. %, it being most preferred if there is no ionic modification at all of the polyurethane urea provided according to the invention.

The polyurethane ureas according to the invention are preferably substantially linear molecules, but they can also be branched. In connection with the present invention, substantially linear molecules are understood as being systems that are readily pre-crosslinked and contain a polycarbonate polyol having a mean hydroxyl functionality of preferably from 1.7 to 2.3, in particular from 1.8 to 2.2, particularly preferably from 1.9 to 2.1. Such systems can still be dispersed to a sufficient degree.

The number-average molecular weight of the polyurethane ureas that are preferably used according to the invention is preferably from 1000 to 200,000, particularly preferably from 5000 to 100,000. The number-average molecular weight is thereby measured against polystyrene as standard in dimethylacetamide at 30° C.

### Polyurethane Ureas

The polyurethane ureas according to the invention are described in detail hereinbelow. The polyurethane ureas according to the invention are prepared by reaction of chain-extension components which comprise at least one polycarbonate polyol component, a polyisocyanate component, a polyoxyalkylene ether component, a diamine and/or amino alcohol component and optionally a polyol component.

The individual chain-extension components are described in detail hereinbelow.

### (a) Polycarbonate Polyol

The polyurethane urea according to the invention comprises units based on at least one hydroxyl-group-containing polycarbonate (polycarbonate polyol).

For the introduction of units based on a hydroxyl-group-containing polycarbonate there are suitable in principle polycarbonate polyols, that is to say polyhydroxy compounds, having a mean hydroxyl functionality of from 1.7 to 2.3, preferably from 1.8 to 2.2, particularly preferably from 1.9 to 2.1. The polycarbonate is accordingly preferably substantially linear and exhibits only slight three-dimensional crosslinking.

Suitable hydroxyl-group-containing polycarbonates are polycarbonates having a molecular weight (molecular weight determined via the OH number; DIN 53240) of preferably from 400 to 6000 g/mol, particularly preferably from 500 to 5000 g/mol, especially from 600 to 3000 g/mol, which are obtainable, for example, by reaction of carbonic acid derivatives, such as diphenyl carbonate, dimethyl carbonate or phosgene, with polyols, preferably diols. There are suitable as such diols, for example, ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetra-ethylene glycol, dipropylene glycol, polypropylene glycols, dibutylene glycol, polybutylene glycols, bisphenol A, tetrabromobisphenol A, but also lactone-modified diols.

The diol component preferably contains from 40 to 100 wt. % hexanediol, preferably 1,6-hexanediol and/or hexanediol derivatives, preferably those which, as well as containing terminal OH groups, contain ether or ester groups, for example products obtained by reaction of 1 mole of hexanediol with at least 1 mole, preferably from 1 to 2 moles, of caprolactone or by etherification of hexanediol with itself to give di- or tri-hexylene glycol. Polyether polycarbonate diols can also be used. The hydroxyl polycarbonates should be substantially linear. They can, however, optionally be branched slightly by the incorporation of polyfunctional components, in particular low molecular weight polyols. Suitable for this purpose are, for example, glycerol, trimethylolpropane, 1,2,6-hexanetriol, 1,2,4-butanetriol, trimethylolpropane, pentaerythritol, quinitol, mannitol, sorbitol, methyl glycoside or 1,3,4,6-dianhydrohexite. Preference is given to polycarbonates based on 1,6-hexanediol as well as co-diols having a modifying action, such as, for example, 1,4-butanediol, or also on ε-caprolactone. Further preferred polycarbonate diols are those based on mixtures of 1,6-hexanediol and 1,4-butanediol.

### (b) Polyisocyanate

The polyurethane urea according to the invention also comprises units based on at least one polyisocyanate.

There can be used as polyisocyanates (b) any aromatic, araliphatic, aliphatic and cycloaliphatic isocyanates known to the person skilled in the art having a mean NCO functionality ≧1, preferably ≧2, on their own or in arbitrary mixtures with one another, it being unimportant whether they have been prepared by phosgene or phosgene-free processes. They can also contain iminooxadiazinedione, isocyanurate, uretdione, urethane, allophanate, biuret, urea, oxadiazinetrione, oxazolidinone, acylurea and/or carbodiimide structures. The polyisocyanates can be used on their own or in arbitrary mixtures with one another.

Preference is given to the use of isocyanates from the group of the aliphatic or cycloaliphatic representatives, these preferably having a carbon skeletal structure (without the NCO groups that are present) of from 3 to 30, preferably from 4 to 20, carbon atoms.

Particularly preferred compounds of component (b) correspond to the above-mentioned type having aliphatically and/or cycloaliphatically bonded NCO groups, such as, for example, bis-(isocyanatoalkyl)ethers, bis- and tris-(isocyanatoalkyl)-benzenes, -toluenes and -xylenes, propane diisocyanates, butane diisocyanates, pentane diisocyanates, hexane diisocyanates (e.g. hexamethylene diisocyanate, HDI), heptane diisocyanates, octane diisocyanates, nonane diisocyanates (e.g. trimethyl-HDI (TMDI), generally in the form of a mixture of the 2,4,4- and 2,2,4-isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis-(isocyanatomethyl)cyclohexane (H₆XDI), 3-isocyanatomethyl-3,5,5-trimethylcyclohexyl isocyanate (isophorone diisocyanate, IPDI), bis-(4-isocyanatocyclohexyl)methane (H₁₂MDI) or bis(isocyanatomethyl)norbornane (NBDI).

Most particularly preferred compounds of component (b) are hexamethylene diisocyanate (HDI), trimethyl-HDI (TMDI), 2-methylpentane-1,5-diisocyanate (MPDI), isophorone diisocyanate (IPDI), 1,3- and 1,4-bis(isocyanatomethyl)cyclohexane (H₆XDI), bis(isocyanatomethyl)norbornane (NBDI), 3(4)-isocyanatomethyl-1-methyl-cyclohexylisocyanate (IMCI) and/or 4,4′-bis(isocyanatocyclohexyl)methane (H₁₂MDI) or mixtures of these isocyanates. Further examples are derivatives of the above diisocyanates having a uretdione, isocyanurate, urethane, allophanate, biuret, iminooxadiazinedione and/or oxadiazinetrione structure with more than two NCO groups.

The amount of constituent (b) in the coating composition to be used according to the invention is preferably from 1.0 to 4.0 mol, particularly preferably from 1.2 to 3.8 mol, especially from 1.5 to 3.5 mol, in each case based on constituent (a) of the coating composition to be used according to the invention.

### (c) Polyoxyalkylene Ethers

The polyurethane urea according to the invention comprises units based on a copolymer of polyethylene oxide and polypropylene oxide. These copolymer units are present in the polyurethane urea as end groups.

Non-ionic hydrophilising compounds (c) are, for example, monohydric polyalkylene oxide polyether alcohols having in the statistical mean from 5 to 70, preferably from 7 to 55, ethylene oxide units per molecule, as are obtainable in a manner known per se by alkoxylation of suitable starter molecules (e.g. in Ullmanns Enzyklopädie der technischen Chemie, 4th Edition, Volume 19, Verlag Chemie, Weinheim p. 31-38).

Suitable starter molecules are, for example, saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3 -ethyl-3-hydroxymethyloxetan or tetrahydrofurfuiyl alcohol, diethylene glycol monoalkyl ethers, such as, for example, diethylene glycol monobutyl ether, unsaturated alcohols such as allyl alcohol, 1,1-dimethylallyl alcohol or oleic alcohol, aromatic alcohols such as phenol, the isomeric cresols or methoxyphenols, araliphatic alcohols such as benzyl alcohol, anisic alcohol or cinnamic alcohol, secondary monoamines such as dimethylamine, diethylamine, dipropylamine, diisopropylamine, dibutylamine, bis-(2-ethylhexyl)-amine, N-methyl- and N-ethyl-cyclohexylamine or dicyclohexylamine as well as heterocyclic secondary amines such as morpholine, pyrrolidine, piperidine or 1H-pyrazole. Preferred starter molecules are saturated monoalcohols. Diethylene glycol monobutyl ether is particularly preferably used as the starter molecule.

The alkylene oxides ethylene oxide and propylene oxide can be used in the alkoxylation reaction in an arbitrary order or also in admixture.

The polyalkylene oxide polyether alcohols are mixed polyalkylene oxide polyethers of ethylene oxide and propylene oxide, the alkylene oxide units of which consist preferably of at least 30 mol %, particularly preferably of at least 40 mol %, ethylene oxide units. Preferred non-ionic compounds are monofunctional mixed polyalkylene oxide polyethers which contain at least 40 mol % ethylene oxide units and not more than 60 mol % propylene oxide units.

The mean molar weight of the polyoxyalkylene ether is preferably from 500 g/mol to 5000 g/mol, particularly preferably from 1000 g/mol to 4000 g/mol, especially from 1000 to 3000 g/mol.

The amount of constituent (c) in the coating composition to be used according to the invention is preferably from 0.01 to 0.5 mol, particularly preferably from 0.02 to 0.4 mol, especially from 0.04 to 0.3 mol, in each case based on constituent (a) of the coating composition to be used according to the invention.

It has been possible to demonstrate according to the invention that polyurethane ureas having end groups based on mixed polyoxyalkylene ethers of polyethylene oxide and polypropylene oxide are particularly suitable for producing coatings having high hydrophilicity. As is shown hereinbelow in comparison with polyurethane ureas terminated only by polyethylene oxide, the coatings according to the invention bring about a markedly smaller contact angle and are accordingly more hydrophilic.

### (d) Diamine or Amino Alcohol

The polyurethane urea according to the invention comprises units based on at least one diamine or amino alcohol.

So-called chain extenders (d) are used in the production of the polyurethane coatings according to the invention. Such chain extenders are di- or poly-amines as well as hydrazides, for example hydrazine, 1,2-ethylenediamine, 1,2- and 1,3-diaminopropane, 1,4-diaminobutane, 1,6-diaminohexane, isophoronediamine, isomeric mixture of 2,2,4- and 2,4,4-trimethylhexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4-diaminodicyclohexylmethane, dimethylethylenediamine, hydrazine, adipic acid dihydrazide, 1,4-bis(aminomethyl)cyclohexane, 4,4′-diamino-3,3′-dimethyldicyclohexylmethane and other (C₁-C₄)-di- and tetra-alkyldicyclohexyl-methanes, for example 4,4′-diamino-3,5-diethyl-3′,5′-diisopropyldicyclohexylmethane.

There come into consideration as diamines or amino alcohols generally low molecular weight diamines or amino alcohols which contain active hydrogen of different reactivity towards NCO groups, such as compounds that contain secondary amino groups in addition to a primary amino group or OH groups in addition to an amino group (primary or secondary). Examples thereof are primary and secondary amines, such as 3-amino-1-methylaminopropane, 3-amino-1-ethylaminopropane, 3-amino-1-cyclohexylaminopropane, 3-amino-1-methylaminobutane, also amino alcohols, such as N-aminoethylethanolamine, ethanolamine, 3-aminopropanol, neopentanolamine and, particularly preferably, diethanolamine.

Constituent (d) of the coating composition to be used according to the invention can be used in the preparation thereof as a chain extender and/or as a chain terminator.

The amount of constituent (d) in the coating composition to be used according to the invention is preferably from 0.05 to 3.0 mol, particularly preferably from 0.1 to 2.0 mol, especially from 0.2 to 1.5 mol, in each case based on constituent (a) of the coating composition to be used according to the invention.

### (e) Polyols

In a further embodiment, the polyurethane urea according to the invention additionally comprises units based on at least one further polyol.

The further low molecular weight polyols (e) used in the synthesis of the polyurethane ureas generally effect stiffening and/or branching of the polymer chain. The molecular weight is preferably from 62 to 500 g/mol, particularly preferably from 62 to 400 g/mol, especially from 62 to 200 g/mol.

Suitable polyols can contain aliphatic, alicyclic or aromatic groups. Examples which may be mentioned here include low molecular weight polyols having up to approximately 20 carbon atoms per molecule, such as, for example, ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxy ethyl ether, bisphenol A (2,2,-bis(4-hydroxyphenyl)propane), hydrogenated bisphenol A (2,2-bis(4-hydroxycyclohexyl)propane), as well as trimethylolpropane, glycerol or pentaerythritol and mixtures of these and optionally also further low molecular weight polyols. It is also possible to use ester diols, such as, for example, α-hydroxybutyl-ε-hydroxy-caproic acid ester, ω-hydroxyhexyl-γ-hydroxybutyric acid ester, adipic acid (β-hydroxyethyl)ester or terephthalic acid bis(β-hydroxyethyl)ester.

The amount of constituent (e) in the coating composition to be used according to the invention is preferably from 0.1 to 1.0 mol, particularly preferably from 0.2 to 0.9 mol, especially from 0.2 to 0.8 mol, in each case based on constituent (a) of the coating composition to be used according to the invention.

(f) Further Amine- and/or Hydroxy-Containing Structural Units (Chain-Extension Component)

The reaction of the isocyanate-containing component (b) with the hydroxy- or amine-functional compounds (a), (c), (d) and optionally (e) is usually carried out while maintaining a slight NCO excess relative to the reactive hydroxy or amine compounds. Residues of isocyanate groups are hydrolysed to amine groups by the dispersion in water. However, it can be important in some cases to block the remaining residue of isocyanate groups before dispersion of the polyurethane.

The polyurethane urea coatings provided according to the invention can therefore also contain chain-extension components (f) which are in each case located at the chain ends and close them off. These structural units are derived on the one hand from monofunctional compounds reactive with NCO groups, such as monoamines, in particular monosecondary amines or monoalcohols.

Examples which may be mentioned here include ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof.

Because the structural units (f) are used in the coatings according to the invention substantially in order to destroy the NCO excess, the required amount is substantially dependent on the amount of the NCO excess and cannot generally be specified.

In a preferred embodiment of the present invention, component (f) is omitted so that the polyurethane urea according to the invention comprises only constituents (a) to (d) and optionally component (e). Furthermore, it is preferred for the polyurethane urea according to the invention to consist of constituents (a) to (d) and optionally component (e), that is to say to contain no other chain-extension components.

### (g) Further Constituents

The polyurethane urea according to the invention can additionally comprise further constituents conventional for the intended purpose, such as additives and fillers. An example thereof are pharmacological active ingredients and additives which promote the release of pharmacological active ingredients (“drug-eluting additives”), as well as medicaments.

Medicaments which can be used in the coatings according to the invention on medical devices are generally, for example, thromboresistant agents, antibiotic agents, antitumour agents, growth hormones, antiviral agents, antiangiogenic agents, angiogenic agents, antimitotic agents, antiinflammatoiry agents, cell-cycle-regulating agents, genetic agents, hormones, as well as their homologues, derivatives, fragments, pharmaceutical salts and combinations thereof.

Specific examples of such medicaments accordingly include thromboresistant (non-thrombogenic) agents or other agents for suppressing an acute thrombosis, stenosis or late restenosis of the arteries, for example heparin, streptokinase, urokinase, tissue plasminogen activator, antithromboxane B₂ agents; anti-B thromboglobulin, prostaglandin E, aspirin, dipyridimol, antithromboxane A₂ agents, murine monoclonal antibody 7E3, triazolopyrimidine, ciprosten, hirudin, ticlopidine, nicorandil, etc. A growth factor can likewise be used as a medicament in order to suppress subintimal fibromuscular hyperplasia at the site of arterial stenosis, or any other desired inhibitor of cell growth at the stenosis site can be used.

The medicament can also consist of a vasodilator in order to counteract vasospasm, for example an antispasmodic agent such as papaverin. The medicament can be a vasoactive agent per se, such as calcium antagonists, or α- and β-adrenergic agonists or antagonists. In addition, the therapeutic agent can be a biological adhesive such as medical grade cyanoacrylate or fibrin, which is used, for example, to bond a tissue flap to the wall of a coronary artery.

The therapeutic agent can also be an antineoplastic agent such as 5-fluorouracil, preferably with a controlled-release carrier for the agent (e.g. for application of an antineoplastic agent that releases continuously in a controlled manner at a tumour site).

The therapeutic agent can be an antibiotic, preferably in combination with a controlled-release carrier for continuous release from the coating of a medical device at a localised source of infection within the body. Similarly, the therapeutic agent can comprise steroids for the purpose of suppressing inflammation in localised tissue or for other reasons.

Specific examples of suitable medicaments include:
(a) heparin, heparin sulfate, hirudin, hyaluronic acid, chondroitin sulfate, dermatan sulfate, keratan sulfate, lytic agents, including urokinase and streptokinase, their homologues, analogues, fragments, derivatives and pharmaceutical salts thereof;
(b) antibiotic agents such as penicillins, cephalosporins, vacomycins, aminoglycosides, quinolones, polymxins, erythromycins; tetracyclines, chloramphenicols, clindamycins, lincomycins, sulfonamides, their homologues, analogues, derivatives, pharmaceutical salts and mixtures thereof;
(c) paclitaxel, docetaxel, immunosuppressants such as sirolimus or everolimus, alkylating agents including mechlorethamine, chlorambucil, cyclophosphamide, melphalan and ifosfamide; antimetabolites including methotrexate, 6-mercapto-purine, 5-fluorouracil and cytarabin; plant alkaloids including vinblastine; vincristine and etoposide; antibiotics including doxorubicin, daunomycin, bleomycin and mitomycin; nitrosurea including carmustin and lomustin; inorganic ions including cisplatin; biological reaction modifiers including interferon; angiostatins and endostatins; enzymes including asparaginase; and hormones including tamoxifen and flutamide, their homologues, analogues, fragments, derivatives, phaimaceutical salts and mixtures thereof; and
(d) antiviral agents such as amantadine, rimantadine, rabavirin, idoxuridine, vidarabin, trifluridine, acyclovir, ganciclovir, zidovudine, phosphonoformates, interferons, their homologues, analogues, fragments, derivatives, pharmaceutical salts and mixtures thereof; and
(e) antiinflammatory agents such as, for example, ibuprofen, dexamethasone or methylprednisolone.

In a preferred embodiment, the coating composition provided according to the invention comprises a polyurethane urea composed of
a) at least one polycarbonate polyol;
b) at least one polyisocyanate;
c) at least one monofunctional mixed polyoxyalkylene ether of polyethylene oxide and polypropylene oxide; and
d) at least one diamine or amino alcohol.

In order to produce surfaces having antifouling properties, the coating compositions according to the invention can comprise antifouling active ingredients known from the prior art. Their presence generally enhances the already outstanding antifouling properties of the surfaces produced with the coating compositions according to the invention themselves.

In a further embodiment of the present invention, the coating composition to be used according to the invention comprises a polyurethane urea composed of
a) at least one polycarbonate polyol;
b) at least one polyisocyanate;
c) at least one monofunctional mixed polyoxyalkylene ether of polyethylene oxide and polypropylene oxide;
d) at least one diamine or amino alcohol; and
e) at least one polyol.

In a further embodiment of the present invention, the coating composition to be used according to the invention comprises a polyurethane urea composed of
a) at least one polycarbonate polyol;
b) at least one polyisocyanate;
c) at least one monofunctional mixed polyoxyalkylene ether of polyethylene oxide and polypropylene oxide;
d) at least one diamine or amino alcohol;
e) at least one polyol; and
f) at least one amine- or hydroxyl-containing monomer which is located at the ends of the polymer chains.

As has already been mentioned, in a most particularly preferred embodiment of the present invention, the polyurethane urea used to produce the preparations to be used according to the invention consists only of constituents (a) to (d) and optionally (e).

Preference is given according to the invention also to polyurethane ureas composed of
a) at least one polycarbonate polyol having a mean molar weight of from 400 g/mol to 6000 g/mol and a hydroxyl functionality of from 1.7 to 2.3, or mixtures of such polycarbonate polyols;
b) at least one aliphatic, cycloaliphatic or aromatic polyisocyanate, or mixtures of such polyisocyanates, in an amount, per mole of polycarbonate polyol, of from 1.0 to 4.0 mol;
c) at least one monofunctional mixed polyoxyalkylene ether of polyethylene oxide and polypropylene oxide, or a mixture of such polyethers, having a mean molar weight of from 500 g/mol to 5000 g/mol in an amount, per mole of polycarbonate polyol, of from 0.01 to 0.5 mol;
d) at least one aliphatic or cycloaliphatic diamine or at least one amino alcohol as so-called chain extenders, or mixtures of such compounds, in an amount, per mole of polycarbonate polyol, of from 0.05 to 3.0 mol;
e) optionally one or more short-chained aliphatic polyols having a molar weight of from 62 g/mol to 500 g/mol in an amount, per mole of polycarbonate polyol, of from 0.1 to 1.0 mol; and
f) optionally amine- or OH-containing structural units which are located at the ends of the polymer chains and close them off.

Further preference is given according to the invention to polyurethane ureas composed of
a) at least one polycarbonate polyol having a mean molar weight of from 500 g/mol to 5000 g/mol and a hydroxyl functionality of from 1.8 to 2.2, or mixtures of such polycarbonate polyols;
b) at least one aliphatic, cycloaliphatic or aromatic polyisocyanate, or mixtures of such polyisocyanates, in an amount, per mole of polycarbonate polyol, of from 1.2 to 3.8 mol;
c) at least one monofunctional mixed polyoxyalkylene ether of polyethylene oxide and polypropylene oxide, or a mixture of such polyethers, having a mean molar weight of from 1000 g/mol to 4000 g/mol in an amount, per mole of polycarbonate polyol, of from 0.02 to 0.4 mol;
d) at least one aliphatic or cycloaliphatic diamine or at least one amino alcohol as so-called chain extenders, or mixtures of such compounds, in an amount, per mole of polycarbonate polyol, of from 0.1 to 2.0 mol;
e) optionally one or more short-chained aliphatic polyols having a molar weight of from 62 g/mol to 400 g/mol in an amount, per mole of polycarbonate polyol, of from 0.2 to 0.9 mol; and
f) optionally amine- or OH-containing structural units which are located at the ends of the polymer chains and close them off

Yet further preference is given according to the invention to polyurethane ureas composed of
a) at least one polycarbonate polyol having a mean molar weight of from 600 g/mol to 3000 g/mol and a hydroxyl functionality of from 1.9 to 2.1, or mixtures of such polycarbonate polyols;
b) at least one aliphatic, cycloaliphatic or aromatic polyisocyanate, or mixtures of such polyisocyanates, in an amount, per mole of polycarbonate polyol, of from 1.5 to 3.5 mol;
c) at least one monofunctional mixed polyoxyalkylene ether of polyethylene oxide and polypropylene oxide, or a mixture of such polyethers, having a mean molar weight of from 1000 g/mol to 3000 g/mol in an amount, per mole of polycarbonate polyol, of from 0.04 to 0.3 mol;
d) at least one aliphatic or cycloaliphatic diamine or at least one amino alcohol as so-called chain extenders, or mixtures of such compounds, in an amount, per mole of polycarbonate polyol, of from 0.2 to 1.5 mol;
e) optionally one or more short-chained aliphatic polyols having a molar weight of from 62 g/mol to 200 g/mol in an amount, per mole of polycarbonate polyol, of from 0.2 to 0.8 mol; and
f) optionally amine- or OH-containing structural units which are located at the ends of the polymer chains and close them off.

The coating compositions to be used according to the invention are applied, for example, to medical devices.

The coating compositions to be used according to the invention in the form of a dispersion can be used to form a coating on a medical device.

The expression “medical device” is to be broadly interpreted within the scope of the present invention. Suitable non-limiting examples of medical devices (including instruments) are contact lenses; cannulas; catheters, for example urological catheters such as urinary catheters or uretral catheters; central venous catheters; venous catheters or inlet and outlet catheters; dilation balloons; catheters for angioplasty and biopsy; catheters used for insertion of a stent, a graft or a cava filter; balloon catheters or other expandable medical devices; endoscopes; laryngoscopes; tracheal devices such as endotracheal tubes, respiratory devices and other tracheal suction devices; bronchoalveolar lavage catheters; catheters used in coronary angioplasty; guide rods, inserters and the like; vessel grafts; pacemaker parts; cochlear implants; dental implant tubes for giving food, drainage tubes; and guide wires.

The coating solutions according to the invention can additionally be used to produce protective coatings, for example for gloves, stents and other implants; extracorporeal blood tubes (blood guide tubes); membranes, for example for dialysis; blood filters; devices for assisting circulation; bandaging material for the care of wounds; urine bags and stoma bags. Also included are implants that contain a medically active agent, such as medically active agents for stents or for balloon surfaces or for contraceptives.

The medical device is usually formed from catheters, endoscopes, laryngoscopes, endotracheal tubes, feeding tubes, guide rods, stents and other implants.

Suitable substrates for the surface to be coated are many materials, such as metals, textiles, ceramics or plastics, the use of plastics being preferred for the production of medical devices.

It has been found according to the invention that medical devices having blood-compatible surfaces that are very hydrophilic, and therefore capable of sliding, can be produced by using aqueous, non-ionically stabilised polyurethane dispersions of the above-mentioned type for coating the medical devices. The above-described coating compositions are preferably obtained in the form of aqueous dispersions and applied to the surface of the medical devices.

As well as being used as a coating for medical devices, the above-described coating compositions can also be used for further technical applications in the non-medical field.

Substrates for applications other than medical coatings are, for example, metals, plastics, ceramics, textiles, leather, wood, paper, coated surfaces of all the mentioned substrates, and glass. The coating materials can be applied directly to the substrate or alternatively to a base coat previously applied to the substrate.

Accordingly, the coatings obtained according to the invention are used to protect surfaces from condensation, to produce surfaces that are easy to clean or self-cleaning. The hydrophilic coatings also reduce the uptake of dirt and prevent the formation of water spots. Possible applications in the external sector are, for example, window panes and skylights, glass façades or Plexiglass roofs. In the internal sector, such materials can be used to coat surfaces in the sanitary field. Further applications are the coating of optical glasses and lenses, such as, for example, spectacle lenses, binocular eyepiece and objective lenses and objective lenses for cameras, or of packaging materials, such as foodstuffs packaging, in order to avoid condensation or the formation of droplets by condensed water.

The coating materials to be used according to the invention are likewise suitable for application to surfaces in contact with water in order to prevent fouling. This effect is also known as an antifouling effect. A very important application of this antifouling effect is in the field of underwater paints for hulls. Hulls without antifouling properties very quickly become overgrown with marine organisms, which, owing to increased friction, leads to a reduction in the possible speed and to higher fuel consumption. The coating materials according to the invention reduce or prevent fouling with marine organisms and prevent the above-mentioned disadvantages of such fouling. Further applications within the field of antifouling coatings are articles for fishing, such as fishing nets, as well as all metal substrates that are used underwater, such as pipelines, drilling platforms, lock chambers and gates, etc. Hulls that have surfaces produced using the coating materials according to the invention, in particular beneath the water line, also have reduced frictional resistance so that ships having such properties either have a reduced fuel consumption or achieve higher speeds. This is of particular interest in the field of leisure craft and yacht building.

A further important field of use of the above-mentioned hydrophilic coating materials is the printing industry. Hydrophobic surfaces can be hydrophilised by the coatings according to the invention and can as a result be printed with polar printing inks or can be applied by means of ink-jet technology.

A further field of application of the hydrophilic coatings used according to the invention are formulations for cosmetic applications.

Active-ingredient-releasing systems based on the hydrophilic coating materials according to the invention are also conceivable outside medical technology, for example for applications in crop protection as a carrier for active ingredients. The coating as a whole can then be regarded as the active-ingredient-releasing system and can be used, for example, to coat seed (grains). As a result of the hydrophilic properties of the coating, the active ingredient that is present is able to emerge in the moist ground and develop its intended action without the germination capacity of the seed being impaired. In the dry state, however, the coating composition binds the active ingredient securely to the seed so that the active ingredient does not become detached, for example when the seed grain is injected into the ground by the sowing machine, as a result of which it could exert undesirable actions, for example on the fauna present (bees are endangered by insecticides which are to prevent the seed in the ground from being attacked by insects).

### Preparation of the Coating Dispersion

The constituents of the coatings described in detail above are generally so reacted that a urea-group-free, isocyanate-functional prepolymer is first prepared by reacting constituents (a), (b), (c) and optionally (e), the ratio of isocyanate groups to isocyanate-reactive groups of the polycarbonate polyol being preferably from 0.8 to 4.0, particularly preferably from 0.9 to 3.8, especially from 1.0 to 3.5.

In an alternative embodiment, it is also possible for constituent (a) first to be reacted separately with the isocyanate (b). Constituents (c) and (e) can then be added and reacted. The remaining isocyanate groups are then generally chain-extended or terminated in an amino-functional manner before, during or after the dispersion in water, the equivalent ratio of isocyanate-reactive groups of the compounds used for chain extension to free isocyanate groups of the prepolymer being preferably from 40 to 150%, particularly preferably from 50 to 120%, especially from 60 to 120% (constituent (d)).

The polyurethane dispersions according to the invention are preferably prepared by the so-called acetone process. In order to prepare the polyurethane dispersion by the acetone process, all or some of constituents (a), (c) and (e), which must not contain any primary or secondary amino groups, and the polyisocyanate component (b) for the preparation of an isocyanate-functional polyurethane prepolymer are generally placed in a reaction vessel and optionally diluted with a solvent that is miscible with water but inert towards isocyanate groups and heated to temperatures in the range from 50 to 120° C. In order to accelerate the isocyanate addition reaction, catalysts known in polyurethane chemistry can be used, for example dibutyltin dilaurate. Preference is given to synthesis without a catalyst.

Suitable solvents are conventional aliphatic, keto-functional solvents such as, for example, acetone, butanone, which can be added not only at the beginning of the preparation but, optionally in portions, also later. Acetone and butanone are preferred. Other solvents, such as, for example, xylene, toluene, cyclohexane, butyl acetate, methoxypropyl acetate, solvents with ether or ester units, can likewise be used and distilled off wholly or partially or remain wholly in the dispersion.

Any constituents of (c) and (e) not added at the beginning of the reaction are then metered in.

In a preferred manner, the prepolymer is prepared without the addition of a solvent and is diluted with a suitable solvent, preferably acetone, only for the chain extension.

In the preparation of the polyurethane prepolymer, the ratio of isocyanate groups to isocyanate-reactive groups is preferably from 0.8 to 4.0, particularly preferably from 0.9 to 3.8, especially from 1.0 to 3.5.

The reaction to the prepolymer takes place partially or completely, but preferably completely. There are thus obtained polyurethane prepolymers containing free isocyanate groups, without a solvent or in solution.

Thereafter, in a further process step, the resulting prepolymer is dissolved with the aid of aliphatic ketones such as acetone or butanone, if this has not yet been carried out or has been carried out only partially.

Possible NH₂—, NH-functional and/or OH-functional components are then reacted with the remaining isocyanate groups. This chain extension/termination can be carried out either in solvents prior to the dispersion, during the dispersion or in water after the dispersion. The chain extension is preferably carried out prior to the dispersion in water.

If compounds according to the definition of (d) having NH₂ or NH groups are used for the chain extension, the chain extension of the prepolymers preferably takes place prior to the dispersion.

The degree of chain extension, that is to say the equivalent ratio of NCO-reactive groups of the compounds used for the chain extension to free NCO groups of the prepolymer, is preferably from 40 to 150%, particularly preferably from 50 to 120%, especially from 60 to 120%.

The aminic components (d) can be used in the process according to the invention individually or in mixtures, optionally in a form diluted in water or solvent, any sequence of addition being possible in principle.

If water or organic solvents are used concomitantly as diluent, the content of diluent is preferably from 70 to 95 wt. %.

The preparation of the polyurethane dispersion from the prepolymers takes place following the chain extension. To this end, the dissolved and chain-extended polyurethane copolymer, optionally with pronounced shear, such as, for example, vigorous stirring, is either introduced into the dispersing water or, conversely, the dispersing water is stirred into the prepolymer solutions. The water is preferably added to the dissolved prepolymer.

The solvent still present in the dispersions after the dispersing step is usually then removed by distillation. Removal during the dispersion is likewise possible.

The solids content of the polyurethane dispersion after synthesis is from 20 to 70 wt. %, preferably from 20 to 65 wt. %. For coating tests, these dispersions can be diluted with water as desired in order to allow the thickness of the coating to be variably adjusted. All concentrations from 1 to 60 wt. % are possible; concentrations in the range from 1 to 40 wt. % are preferred.

Any desired layer thicknesses can be achieved, such as, for example, from several 100 nm to several 100 μm, larger and smaller thicknesses also being possible within the scope of the present invention.

The polyurethane materials for coating the medical devices can be diluted to any desired value by diluting the aqueous dispersions according to the invention with water. In addition, thickeners can be added in order to enable the viscosity of the polyurethane dispersions to be increased if required. Further additives, such as, for example, antioxidants, buffer materials for adjusting the pH value or pigments, are also possible. In addition, further additives, such as agents for improving handle, colourings, mattifying agents, UV stabilisers, light stabilisers, hydrophobising agents, hydrophilising agents and/or flow improvers, can optionally be used.

Starting from these dispersions, medical coatings are then produced by the processes described hereinbefore.

It has been found according to the invention that the resulting coatings on medical devices differ according to whether the coating is produced from a dispersion or from a solution.

The coatings according to the invention on medical devices have advantages when they are obtained from dispersions of the above-described coating compositions because dispersions of the coating systems according to the invention yield coatings on the medical devices that do not contain organic solvent residues, that is to say are generally toxicologically harmless, and at the same time result in a more pronounced hydrophilicity, which manifests itself, for example, in a small contact angle. Reference is made in this connection to the tests and comparison tests described hereinbelow.

The medical devices can be coated with the hydrophilic polyurethane dispersions according to the invention by means of various processes. Suitable coating techniques for this purpose are, for example, knife coating, printing, transfer coating, spraying, spin coating or dipping.

The aqueous polyurethane dispersions used as starting material for the production of the coatings can be prepared by any desired processes, but the acetone process described above is preferred.

Many different substrates can be coated, such as metals, textiles, ceramics and plastics. Preference is given to the coating of medical devices manufactured from metals or plastics. Examples of metals which may be mentioned include: medical stainless steel or nickel-titanium alloys. Medical devices that are to be coated can consist of different polymer materials, alone or in combination, such as, for example, polyamide, polystyrene, polycarbonate, polyethers, polyesters, polyvinyl acetate, natural and synthetic rubbers, block copolymers of styrene and unsaturated compounds such as ethylene, butylene and isoprene, polyethylene or copolymers of polyethylene and polypropylene, silicone, polyvinyl chloride (PVC) and polyurethanes. For the purpose of better adhesion of the hydrophilic polyurethane to the medical device, further suitable coatings (primers, adhesion promoters) can be applied as undercoat before the hydrophilic coating materials are applied.

In addition to the hydrophilic properties for improving the sliding capacity, the coatings produced according to the invention are also distinguished by high blood compatibility. As a result, it is also particularly advantageous to work with these coatings especially in contact with blood. Compared with polymers of the prior art, the materials have a reduced clotting tendency in contact with blood.

The advantages of the catheters coated according to the invention with the hydrophilic polyurethane coatings are demonstrated in the following examples by means of comparison tests.

### EXAMPLES

The NCO content of the resins described in the examples and comparison examples was determined by titration according to DIN EN ISO 11909.

The solids contents were determined according to DIN-EN ISO 3251. 1 g of polyurethane dispersion was dried to constant weight (15-20 minutes) at 115° C. by means of an infra-red dryer.

The mean particle sizes of the polyurethane dispersions were measured with the aid of a High Performance Particle Sizer (HPPS 3.3) from Malvern Instruments.

Unless stated otherwise, the amounts in % are to be understood as being wt. % and are based on the resulting aqueous dispersion.

Substances and Abbreviations Used:
Desmophen® C2200: Polycarbonate polyol, OH number 56 mg KOH/g, number-average molecular weight 2000 g/mol (Bayer MaterialScience AG, Leverkusen, DE)
Desmophen® C1200: Polycarbonate polyol, OH number 56 mg KOH/g, number-average molecular weight 2000 g/mol (Bayer MaterialScience AG, Leverkusen, DE)
Desmophen® XP 2613 Polycarbonate polyol, OH number 56 mg KOH/g, number-average molecular weight 2000 g/mol (Bayer MaterialScience AG, Leverkusen, DE)
PolyTHr 2000: Polytetramethylene glycol polyol, OH number 56 mg KOH/g, number-average molecular weight 2000 g/mol (BASF AG, Ludwigshafen, DE)
Polyether® LB 25: (mono functional polyether based on ethylene oxide/propylene oxide, number-average molecular weight 2250 g/mol, OH number 25 mg KOH/g (Bayer MaterialScience AG, Leverkusen, DE)

### Example 1

### Preparation of a Polyurethane Urea Dispersion According to the Invention

277.2 g of Desmophen® C 2200, 33.1 g of Polyether LB 25 and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 110° C. After 3 hours 40 minutes, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 15 minutes. Dispersion was then carried out in the course of 15 minutes by addition of 590 g of water. The solvent was then removed by distillation in vacuo. A storage-stable polyurethane dispersion having a solids content of 41.5% and a mean particle size of 164 nm was obtained.

### Example 2

### Preparation of a Polyurethane Urea Dispersion According to the Invention

269.8 g of Desmophen® C 2200, 49.7 g of Polyether LB 25 and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 100° C. After 21.5 hours, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 41.3% and a mean particle size of 109 nm was obtained.

### Example 3

### Preparation of a Polyurethane Urea Dispersion According to the Invention

277.2 g of Desmophen® C 1200, 33.1 g of Polyether LB 25 and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 110° C. After 2.5 hours, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 40.4% and a mean particle size of 146 nm was obtained.

### Example 4

### Preparation of a Polyurethane Urea Dispersion According to the Invention

282.1 g of Desmophen® C 2200, 22.0 g of Polyether LB 25 and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 110° C. After 21.5 hours, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 41.7% and a mean particle size of 207 nm was obtained.

### Example 5

### Preparation of a Polyurethane Urea Dispersion According to the Invention

269.8 g of Desmophen® XP 2613, 49.7 g of Polyether LB 25 and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 110° C. After 70 minutes, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 41.2% and a mean particle size of 112 nm was obtained.

### Example 6

### Preparation of a Polyurethane Urea Dispersion According to the Invention

249.4 g of Desmophen® C 2200, 33.1 g of Polyether LB 25, 1.9 g of trimethylolpropane and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 110° C. After 4 hours 20 minutes, the theoretical NCO value had been achieved. The finished prepolymer was dissolved at 50° C. in 720 g of acetone, and then a solution of 3.3 g of ethylenediamine in 16 g of water was metered in at 40° C. in the course of 10 minutes. The after-stirring time was 15 minutes. Dispersion was then carried out in the course of 15 minutes by addition of 590 g of water. The solvent was then removed by distillation in vacuo. A storage-stable polyurethane dispersion having a solids content of 38.9% and a mean particle size of 144 nm was obtained.

### Example 7

282.1 g of Desmophen® XP 2613, 22.0 g of Polyether LB 25 and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 110° C. After 70 minutes, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 38.3% and a mean particle size of 215 nm was obtained.

### Example 8

### Preparation of a Polyurethane Urea Dispersion as Comparison Product to Example 1 According to the Invention. Desmophen® C2200 is Replaced by PolyTHF 2000

277.2 g of PolyTHF 2000, 33.1 g of Polyether LB 25 and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 110° C. After 18 hours, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 40.7% and a mean particle size of 166 nm was obtained.

### Example 9

### Preparation of a Polyurethane Urea Dispersion as Comparison Product to Example 2 According to the Invention. Desmophen® C2200 is Replaced by PolyTHF 2000

269.8 g of PolyTHF 2000, 49.7 g of Polyether LB 25 and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 100° C. After 17.5 hours, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 41.6% and a mean particle size of 107 nm was obtained.

### Example 10

### Preparation of a Polyurethane Urea Dispersion as Comparison Product to Example 4 According to the Invention. Desmophen® C2200 is Replaced by PolyTHE 2000

282.1 g of PolyTHF 2000, 22.0 g of Polyether LB 25 and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 110° C. After 21.5 hours, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 37.5% and a mean particle size of 195 nm was obtained.

### Example 11

### Production of Coatings and Measurement of the Static Contact Angle

The coatings for measurement of the static contact angle were produced by means of a spin coater (RC5 Gyrset 5, Karl Siiss, Garching, Germany) on glass slides having a size of 25×75 mm To this end, a slide was clamped on the sample plate of the spin coater and covered homogeneously with approximately from 2.5 to 3 g of aqueous undiluted polyurethane dispersion. A homogeneous coating was obtained by rotating the sample plate for 20 seconds at 1300 revolutions per minute, which coating was dried for 15 minutes at 100° C. and then for 24 hours at 50° C. The resulting coated slides were immediately subjected to a contact angle measurement.

A static contact angle measurement is carried out on the resulting coatings on the slides. 10 drops of Millipore water are applied to the sample by means of a video-based contact angle measuring device OCA20 from Dataphysics with computer-controlled syringes, and their static wetting angle was measured. The static charge (if present) on the sample surface was removed beforehand by means of an antistatic hair-dryer.

TABLE 1
Static contact angle measurements

| PU FILM | CONTACT ANGLE [°] |
|---|---|
| Example 1 | <10 |
| Example 2 | 11 |
| Example 3 | 14 |
| Example 4 | 20 |
| Example 5 | 14 |
| Example 6 | 26 |
| Example 7 | 41 |
| Comparison Example 8 | 66 |
| Comparison Example 9 | 62 |
| Comparison Example 10 | 77 |

As Table 1 shows, the polycarbonate-containing coatings of Examples 1 to 7 according to the invention produce extremely hydrophilic coatings with static contact angles ≦45° . The coatings of Examples 1 to 6 give extraordinarily hydrophilic coatings with static contact angles <30° . The polyTHF-containing coatings of Comparison Examples 7 to 10, on the other hand, are substantially more non-polar, although the compositions of these coatings are otherwise identical with those of Examples 1, 2 and 4.

In addition, data disclosed in “Evaluation of a poly(vinylpyrrolidone)-coated biomaterial for urological use”; M. M. Tanney, S. P. Gorman, Biomaterials 23 (2002), 4601-4608 show that the contact angle of polyurethane is about 97° and that of PVP-coated polyurethane is about 50° .

### Example 12

### Measurement of Coagulation Parameters

A film for studies in contact with blood was produced from the polyurethane dispersion of Example 1 on glass by spin coating. The sample surface was placed in an autoclaved incubation chamber and incubated with 1.95 ml of blood. The exact test arrangement is described in U. Streller et al. J. Biomed. Mater. Res B, 2003, 66B, 379-390.

The venous blood required for the test was taken via a 19 G cannula from a male donor who had not taken any medication for at least 10 days. Coagulation was inhibited by addition of heparin (2 iU/ml). The blood so prepared was then introduced into the incubation chamber, pre-tempered at 37° C., containing the polyurethane surface and incubated for 2 hours at 37° C. with constant rotation of the chamber. Glass and polytetrafluoroethylene (PTFE) were used as comparison materials. Glass is a highly activating surface for blood coagulation, while PTFE is a polymer which is an acceptable material for many applications (see U. Streller et al. J. Biomed. Mater. Res B, 2003, 66B, 379-390).

When incubation had taken place, three parameters were measured:
thrombin-antithrombin complex (Enzygnost TAT micro, Dade Behring GmbH, Marburg, Germany)
platelet factor 4 (ELISA PF 4 complete kit from Haemochrom Diagnostica GmbH, Essen, Germany)
The thrombocyte reduction was measured in EDTA anticoagulated blood by means of an automatic cell counting system (AcTdiff from Coulter, Krefeld, Germany).

TABLE 2
Thrombin-antithrombin complex

| | Thrombin-antithrombin complex |
|---|---|
| Surface | (μg/mL) |
| Polyurethane of Example 1 | 27.7 |
| PTFE | 33.4 |

TABLE 3
Platelet factor 4

| | Thrombin-antithrombin complex |
|---|---|
| Surface | (IU/mL) |
| Polyurethane of Example 1 | 29.7 |
| Glass | 377.2 |
| PTFE | 59.2 |

TABLE 4
Thrombocyte reduction in the blood

| | Thrombocyte count |
|---|---|
| Surface | (% reduction) |
| Polyurethane of Example 1 | −0.3 |
| Glass | 17.9 |
| PTFE | 5.7 |

All three measured blood parameters show that the hydrophilic polyurethane of Example 1 activates coagulation only very moderately. Thrombin-antithrombin complex, as a measure of the activation of the intrinsic coagulation cascade, shows that the polyurethane itself, compared with PTFE, which is regarded as having very high blood compatibility, produces lower values and accordingly causes even less activation.

Platelet factor 4 is a marker for the activation of thrombocytes. Even this cellular part of coagulation is activated to only a small degree by the hydrophilic polyurethane. PTFE, which has high blood compatibility, brings about higher activation. The thrombocyte reduction, too, is marked for glass and also PTFE, which means that some of the thrombocytes become attached to these surfaces. In the case of the hydrophilic polyurethane of Example 1, on the other hand, scarcely any reduction is detectable.

### Example 13

Synthesis of an aqueous dispersion with terminal polyethylene oxide units as comparison material to the examples according to the invention, in which a polyurethane terminated by a copolymer of ethylene oxide and propylene oxide is used. Polyether LB 25 used within the scope of the invention is replaced in this example by equal molar amounts of a comparable pure polyethylene oxide ether.

277.2 g of Desmophen® C 2200, 29.4 g of polyethylene glycol 2000 monomethyl ether (source: Fluka, CAS No. 9004-74-4) and 6.7 g of neopentyl glycol were placed in a reaction vessel at 65° C. and homogenised for 5 minutes by stirring. To this mixture there were added at 65° C., in the course of 1 minute, first 71.3 g of 4,4′-bis(isocyanatocyclohexyl)methane (H₁₂MDI) and then 11.9 g of isophorone diisocyanate. The mixture was heated to 110° C. After 35 minutes, the theoretical NCO value had been achieved. The finished prepolymer was dissolved at 50° C. in 711 g of acetone, and then a solution of 4.8 g of ethylenediamine in 16 g of water was metered in at 40° C. in the course of 10 minutes. The after-stirring time was 5 minutes. Dispersion was then carried out in the course of 15 minutes by addition of 590 g of water. The solvent was then removed by distillation in vacuo. A storage-stable polyurethane dispersion having a solids content of 40.0% and a mean particle size of 130 rim was obtained.

As described in Example 11, a coating was produced on glass by spin coating and the static contact angle of the coating was determined. A static contact angle of 45° was obtained. Comparison of this value with the value for the coating of Example 1 (<10° , see Table 1 in Example 11) shows that the use of the mixed polyethylene oxide-polypropylene oxide monool LB 25 permits a markedly lower contact angle and accordingly more hydrophilic coatings as compared with the pure polyethylene oxide monool.

### Example 14

### Synthesis of the Polyurethane Urea Polymer of Example 1 According to the Invention as a Comparison Example in Organic Solution

To a mixture of 277.2 g of Desmophen® C 2200, 33.1 g of LB 25, 6.7 g of neopentyl glycol there are added at 60° C. 71.3 g of 4,4′-bis(isocyanatocyclohexyl)methane (H₁₂MDI) and 11.9 g of isophorone diisocyanate. The mixture was heated to 110° C. and reacted to a constant NCO content of 2.4 um. The mixture was allowed to cool and was diluted with 475 g of toluene and 320 g of isopropanol. A solution of 4.8 g of ethylenediamine in 150 g of 1-methoxy-2-propanol was added at room temperature in the course of. When the addition was complete, after-stirring was carried out for 2 hours. 1350 g of a 30.2% polyurethane urea solution in toluene/isopropanol/1-methoxy-2-propanol having a viscosity of 607 mPas at 23° C. were obtained.

As described in Example 11, a coating was produced on glass by spin coating and the static contact angle of the coating was determined. A static contact angle of 27° was obtained. Comparison of this value with the value for the coating of Example 1 (<10°, see Table 1 in Example 11), a coating that is structurally similar but dispersed in water, shows that the coatings from aqueous dispersion give more hydrophilic coatings as compared with coatings obtained from corresponding solutions.

1.-16. (canceled)

17. A coating composition in the foun of a dispersion comprising a polyurethane urea which
(1) is terminated by a copolymer unit of polyethylene oxide and polypropylene oxide, and
(2) comprises at least one hydroxyl-group-containing polycarbonate polyol.

18. The coating composition according to claim 17, wherein the polyurethane comprises units based on at least one aliphatic, cycloaliphatic or aromatic isocyanate.

19. The coating composition according to claim 17, wherein the polyurethane urea has a maximum ionic modification of 2.5 wt. %.

20. The coating composition according to claim 17, wherein the coating composition comprises a polyurethane urea comprising
a) at least one polycarbonate polyol having a mean molar weight of from 400 g/mol to 6000 g/mol and a hydroxyl functionality of from 1.7 to 2.3, or mixtures of such polycarbonate polyols;
b) at least one aliphatic, cycloaliphatic or aromatic polyisocyanate, or mixtures of such polyisocyanates, in an amount, per mole of polycarbonate polyol, of from 1.0 to 4.0 mol;
c) at least one monofunctional mixed polyoxyalkylene ether of polyethylene oxide and polypropylene oxide, or a mixture of such polyethers, having a mean molar weight of from 500 g/mol to 5000 g/mol in an amount, per mole of polycarbonate polyol, of from 0.01 to 0.5 mol;
d) at least one aliphatic or cycloaliphatic diamine or at least one amino alcohol, or mixtures of such compounds, in an amount, per mole of polycarbonate polyol, of from 0.05 to 3.0 mol;
e) optionally one or more short-chained aliphatic polyols having a molar weight of from 62 g/mol to 500 g/mol in an amount, per mole of polycarbonate polyol, of from 0.1 to 1.0 mol; and
f) optionally amine- or OH-containing structural units which are located at the ends of the polymer chains and close them off.

21. A method of preparing a coated substrate with the coating composition according to claim 20, comprising the steps of:
A) preparing a coating composition which comprises the steps of
(I) placing constituents (a), (b), (c) and optionally (e) in a reaction vessel and optionally diluting them with a solvent that is miscible with water but inert towards isocyanate groups;
(II) heating the composition obtainable from (I) to temperatures in the range of from 50 to 120° C.;
(III) metering in any constituents of (c) and optionally (e) not added at the beginning of the reaction;
(IV) dissolving the resulting prepolymer from (III) with the aid of aliphatic ketones;
(V) adding constituent (d) for chain extension:
(VI) adding water to form a dispersion; and
(VII) removing the aliphatic ketone, and
B) coating a substrate with the coating composition obtained according to (A).

22. The method according to claim 21, wherein the aliphatic ketone is removed in step (VIII) by distillation.

23. The method according to claim 21, wherein after coating the substrate, the substrate is easy to clean or self-cleaning.

24. The method according to claim 21, wherein the substrate is glass, optical glass, or a lens.

25. The method according to claim 21, wherein the coated substrate is used in the sanitary field.

26. The method according to claim 21, wherein the substrate is a packaging material.

27. The method according to claim 21, wherein after coating, the substrate reduces fouling of the coated substrate.

28. The method according to claim 21, wherein the substrate is an over- and under-water substrate, and wherein after coating, the substrate has a reduced frictional resistance towards water.

29. The method according to claim 21, further comprising printing on the substrate after the substrate is coated.

30. A formulation for cosmetic applications comprising the coating composition according to claim 17.

31. An active-ingredient-releasing system for coating seeds comprising the coating composition according to claim 17, wherein the substrate is a seed.

32. A glass, optical glass, or lens comprising the coating composition according to claim 17.

33. A packaging material comprising the coating composition according to claim 17.

34. A seed comprising the coating composition according to claim 17.

35. A medical device comprising the coating composition according to claim 17.

36. A substrate in the non-medical field comprising the coating composition according to claim 17.
